(19) United States
(12) Patent Application Publication
(10) Pub. No.: US 2018/0271939 A1
(43) Pub. Date: Sep. 27, 2018

(54) GENETIC METHOD TO KILL CANCER CELLS BY SUFFOCATION

(71) Applicants: Milton J. Silverman, JR., San Diego, CA (US); Franco A. Ferrari, La Jolla, CA (US)

(72) Inventors: Milton J. Silverman, JR., San Diego, CA (US); Franco A. Ferrari, La Jolla, CA (US)

(21) Appl. No.: 15/927,007

(22) Filed: Mar. 20, 2018

Related U.S. Application Data
(60) Provisional application No. 62/476,528, filed on Mar. 24, 2017.

(51) Int. Cl. A61K 38/17; A61K 48/00; A61P 35/00

(52) U.S. Cl. CPC A61K 38/1767; A61K 48/0075; C12N 15/88; A61P 35/00; A61K 48/005

(57) ABSTRACT

A genetic method to kill cancer by suffocation is presented. The invention involves directly administering a bacterial plasmid in the form of an expression vector to cancer cells or tissue in a patient with cancerous growth. The plasmid is in the form of an expression vector with a nucleic acid encoding the silk protein gene.

### CROSS-REFERENCE TO RELATED APPLICATIONS

The present application claims the benefit of U.S. Provisional Application Ser. No. 62/476,528, filed on Mar. 24, 2017, specification of which is herein incorporated by reference for completeness of disclosure.

### BACKGROUND OF THE INVENTION

### Field of the Invention

Embodiments of the invention relates to the field of gene therapy. More specifically, the invention relates to a genetic method to kill cancer by suffocation.

### Description of the Related Art

Cancer cells owe their quasi-perpetual existence to a variety of mechanisms that provide selective survival advantages over normal cells. One of these mechanisms is the activation of telomerase which confers to transformed cells the immortality trait. Like any mammalian cells, cancer cells require nutrients for their survival which include oxygen, sugars, and proteins. Unlike normal cells, cancer cells have an accelerated metabolism and to sustain their growth and proliferation they are far more dependent on nutrient supply. To accomplish this, cancer cells have developed a sophisticated mechanism to increase blood and lymph supply called neoangiogenesis and neolymphangogenesis, respectively. Therefore, during their growth, cancer compensates for its greater demand for nutrients by increasing afferent blood vessels and lymphatics.

Cancer is a disease characterized by uncontrolled growth and spread of abnormal cells. Cancer can be caused by both external factors (e.g., tobacco, infectious agents, chemicals, and radiation) or internal factors (inherited mutations, hormones, metabolisms defects, and immune conditions). According to the American Cancer Society, in 2017, there will be an estimated 1,688,780 new cancer cases diagnosed and 600,920 cancer deaths in the US.

Various methods have been tried during this and the last century to kill cancer cells, including cutting, poisoning and burning.

Traditionally, cancer is treated with a combination of surgery, radiation and chemotherapy (cut, burn and poison). However, for a large number of cancer types these traditional approaches have proven to be unable to return patients to a normal life. More recently, immunotherapies (immune checkpoint inhibitors and CAR-T cells) have spurred new waves of excitement as they have succeeded in providing a cure or a prolonged survival in a number of instances. However, the successes of immunotherapy are for the time being limited to selected type of cancers and are effective in a limited (˜30%) proportion of patients in the case of immune checkpoint inhibitors or are exorbitantly expensive in the case of CAR-T. Vaccines against cancer have yet to prove they can attain a reasonable degree of success through controllable mechanisms.

None of the traditional methods of curing cancer includes the new disruptive method: killing cancer through a “suffocation” mechanism whereby cancer cells are made to build a protein mesh that ultimately blocks the cancer cells most basic metabolic activities such as import of nutritional elements and export of metabolic decay products. This method which has never been sought or attempted before offers a simple solution to intractable cancers.

### BRIEF SUMMARY OF THE INVENTION

One or more embodiments of the invention are directed at a genetic method to kill cancer by suffocation. The invention comprises inducing cancer cells to create an encasement around themselves that would lead to their progressive dysfunction and inability to proliferate. The method comprises making cancer cells build a protein mesh that would ultimately block their most basic metabolic activities such as import of nutritional elements and export of metabolic decay products. Since a cancer cell has to breathe, eat, and defecate, if the cell is encased with an impermeable membrane, it will die.

In one or more embodiments, the impermeable membrane comprises material that will not provoke an immune response, e.g. silk. The cancer cells would be programmed to manufacture silkworm protein (silk), a protein composed of repetitive sequences spontaneously forming non-immunogenic polymers resistant to degradation.

In another embodiment, cancer cells are genetically modified with the gene of the silk protein which itself is associated with a transmembrane domain to enable anchoring of the secreted silk protein to the surface of the cancer cell, facilitating the formation of a matrix coating the surface of the cancer cell.

In another embodiment, cancer cells are genetically modified with the gene coding for the silk protein chimerized with the peptidic motif comprising the three amino acid arg-gly-asp (RGD). The RGD motif is a ligand for the alpha-v-beta-3-integrin which is expressed on the cell surface in many types of cancer. The RGD motif causes the newly secreted silk protein to tether to the surface of cancer cells, hence facilitating the formation of a matrix which will encase the cancer cell. The advantage of using silk is that it is a non-immunogenic material.

In another embodiment, the cancer cells are genetically modified with the gene coding for the silk protein, whereas the gene is associated with both the transmembrane domain and the RGD coding motif. The secreted silk protein is not only retained on the surface via the transmembrane domain but its free end is also tethered to the cell surface via the RGD motif.

The methods of the present invention provide a rational and effective approach to treat cancer by causing its demise by effectively reducing or completely eliminating nutrient supply vehicle through blood vessels and lymphatics. In addition, this approach also depletes nutrients present in the extracellular space, such as minerals, like iron, which promotes cell growth.

It has already been demonstrated that mammalian cells can be transfected with the gene coding for spider silk fibers and produce silk fibers through a process which mimics the synthetic machinery present in the ampullate gland of orb-weaver spinning spiders, or in silk worms.

One or more embodiments of the present invention are based on transfecting cancer cells with a silk gene of predefined size but not bigger than 60 kilodaltons.

One or more embodiments of the present invention comprises a modification of the silk gene to be physically linked with a transmembrane domain.

One or more embodiments of the present invention comprises a modification of the silk gene to be physically linked with one or multiple repeats of the RGD motif.

One or more embodiments of the present invention comprises a modification of the silk gene to be physically linked with a transmembrane domain at one end and with one or more RGD repeats at the other.

### BRIEF DESCRIPTION OF THE DRAWING

The above and other aspects, features and advantages of the invention will be more apparent from the following more particular description thereof, presented in conjunction with the following drawing wherein:

FIG. 1 is an illustration of the process by which the silk protein encases and kills cancer cells in accordance with one or more embodiments of the present invention.

### DETAILED DESCRIPTION

The present invention comprising a genetic method to kill cancer by suffocation will now be described. In the following exemplary description numerous specific details are set forth in order to provide a more thorough understanding of embodiments of the invention. It will be apparent, however, to an artisan of ordinary skill that the present invention may be practiced without incorporating all aspects of the specific details described herein. Furthermore, although steps or processes are set forth in an exemplary order to provide an understanding of one or more systems and methods, the exemplary order is not meant to be limiting. One of ordinary skill in the art would recognize that the steps or processes may be performed in a different order, and that one or more steps or processes may be performed simultaneously or in multiple process flows without departing from the spirit or the scope of the invention. In other instances, specific features, quantities, or measurements well known to those of ordinary skill in the art have not been described in detail so as not to obscure the invention. It should be noted that although examples of the invention are set forth herein, the claims, and the full scope of any equivalents, are what define the metes and bounds of the invention.

For a better understanding of the disclosed embodiment, its operating advantages, and the specified object attained by its uses, reference should be made to the accompanying drawings and descriptive matter in which there are illustrated exemplary disclosed embodiments. The disclosed embodiments are not intended to be limited to the specific forms set forth herein. It is understood that various omissions and substitutions of equivalents are contemplated as circumstances may suggest or render expedient, but these are intended to cover the application or implementation.

The term “first”, “second” and the like, herein do not denote any order, quantity or importance, but rather are used to distinguish one element from another, and the terms “a” and “an” herein do not denote a limitation of quantity, but rather denote the presence of at least one of the referenced item.

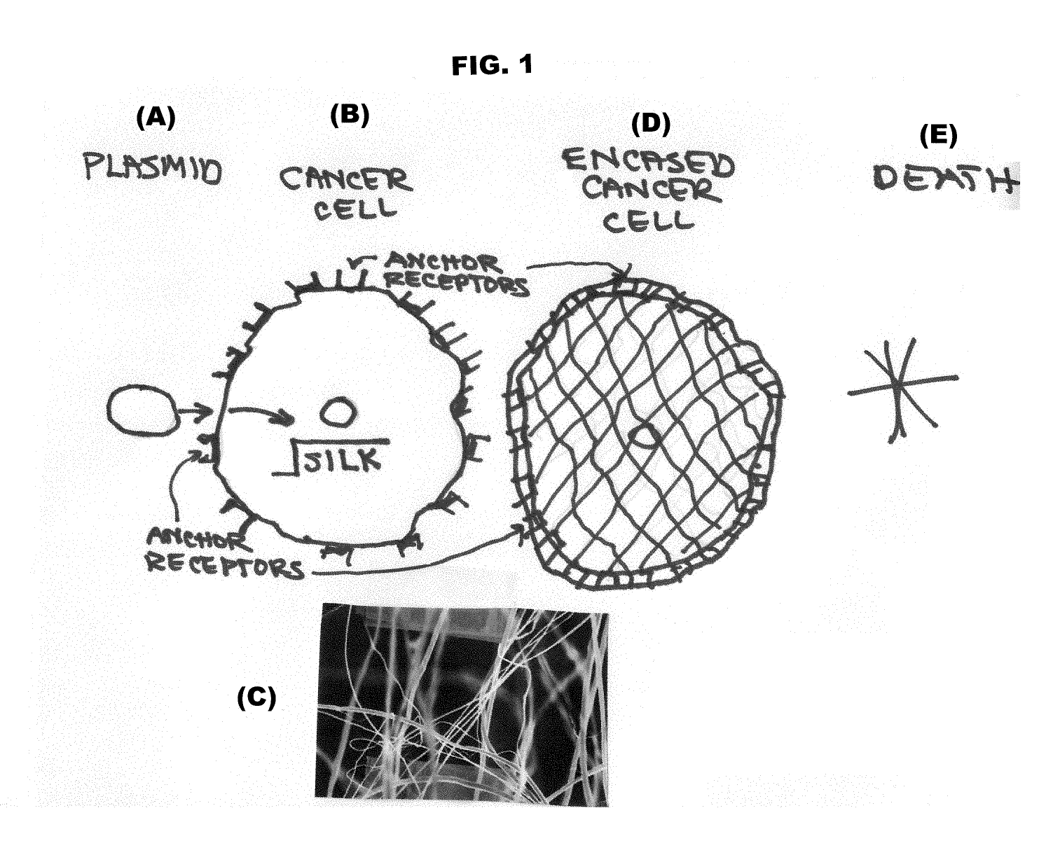

One or more embodiments of the invention consists of genetically modifying cancer cells so that they will produce a matrix which will encase the cancer cells, blocking the transport of nutrients and support, by encasing, suffocating, and strangling the cancer cells. As illustrated in FIG. 1, the plasmid encoding the silk gene protein (A) is introduced into the cancer cell (B). The cell then produces the silk fibers (C) which form a matrix that encases the cancer cell (D) thereby suffocating, and strangling the cancer cell leading to its death (E).

Silk is produced by spiders and worms. It has an unusual property. While it is being produced inside the spider or worm's body, it is permeable. When it goes outside, it suddenly becomes impermeable. In addition, it also has elasticity. The architecture of silk is such that once outside the body, the strands have a tendency to “find” other strands and interlock.

In the twentieth century, the military was interested in whether silk fibers could be used as a cover or wrapper on airplanes to make them stealthy. Investigators found that a mammalian cell could be used to produce silk fibers. This means that cells inside the human body can be engineered to produce silk.

Once these fibers go outside the cell, they become impermeable. The head and tail of each strand of fiber are manipulated so that the head will tie on one site on the cell surface and the tail will anchor on another. This will create an impermeable membrane around the cell resulting in the death of the cell.

In one or more embodiments, cancer cells are genetically modified with the gene of the silk protein whose enforced synthesis secretion form a matrix that will encase the cancer cell thereby depriving it of its three most essential functions, to eat, breathe and defecate.

In another embodiment, the gene of the silk protein is associated with a transmembrane domain to enable anchoring of the secreted silk protein to the surface of the cancer cell, facilitating the formation of a matrix coating the surface of the cancer cell.

In another embodiment, cancer cells are genetically modified with the gene coding for the silk protein chimerized with the peptidic motif comprising the three amino acid arg-gly-asp (RGD). The RGD motif is a ligand for the alpha-v-beta-3-integrin which is expressed on the cell surface in many types of cancer. The RGD motif causes the newly secreted silk protein to tether to the surface of cancer cells, hence facilitating the formation of a matrix which will encase the cancer cell. The advantage of using silk is that it is a non-immunogenic material.

In another embodiment, the gene is associated with both the transmembrane domain and the RGD coding motif. The secreted silk protein is not only retained on the surface via the transmembrane domain but its free end is also tethered to the cell surface via the RGD motif.

One or more embodiments of the present invention comprises a modification of the silk gene to be physically linked with a transmembrane domain as described by Blum et al., (Journal of Biological Chemistry, 1993).

One or more embodiments of the present invention comprises a modification of the silk gene to be physically linked with one or multiple repeats of the RGD motif.

One or more embodiments of the present invention comprises a modification of the silk gene to be physically linked with a transmembrane domain at one end and with one or more RGD repeats at the other.

### Expression Vectors

The silk coding gene, with or without the modifications indicated above, is preferably included and propagated within a bacterial plasmid in the form of an expression vector containing an origin of replication and a selection marker such as the neomycin resistance gene. Embodiments of the invention use the expression vector to introduce the silk coding gene into the target cancerous cell, thereby using the cell's protein synthesis mechanism to produce the silk protein encoded by the gene.

### Method of Delivery

In one or more embodiments, a bacterial plasmid comprising an expression vector encompassing the silk gene with or without the modifications described above will be administered by direct injection into a tumor that is easily accessible.

In another embodiment, the bacterial plasmid comprising an expression vector encompassing the silk gene with or without the modifications described above can be injected intravenously, for example, in the portal vein to target the liver.

In another embodiment, the bacterial plasmid comprising an expression vector encompassing the silk gene with or without the modification described above can be injected systemically in the form of encapsulated material such as liposomes, exosomes, or any type of chemically synthesized or biologically derived small vesicles.

### Mechanism of Action

In its simplest form, in accordance with embodiments of the present invention, when a cancer cell is transfected with a gene coding from the bacterial plasmid encompassing the silk gene, with or without the modifications described above, it will over time synthesize and secrete silk fibers that will form a polymer encasing the cancer cell and cause its death.

In the case where cancerous tissues are the target of the therapeutic intervention, the invention requires that a sufficient number of cells are effectively transfected with the bacterial plasmid encompassing the silk gene, with or without the modifications described above, to reach the desired therapeutic effect.

One or more embodiments of the present invention comprises programming the cancer cell to manufacture silkworm protein (silk), a protein composed of repetitive sequences spontaneously forming non-immunogenic polymers that are very resistant to degradation. Based on isolated reports showing that silk can be produced in mammalian cells, the invention uses a gene in the form of plasmid DNA that is ideally suited for transcription and translation in human cancer cells.

In one or more embodiments, a plasmid vector (i.e. a piece of bacterial DNA) with the silkworm protein gene is created. The plasmid DNA used in the embodiment discussed herein is referred to as pPT0187 (polymer abbreviated: SELPF (Silk₈Elastin17F)ₙ). The sequence was published as plasmid pPT0183, along with other polymers, in U.S. Pat. No. 5,641,648, titled “Methods for preparing synthetic repetitive DNA,” which is herein incorporated by reference. The construction of synthetic protein polymer was published in U.S. Pat. No. 5,514,581, titled “Functional recombinantly prepared synthetic protein polymer,” which is also herein incorporated by reference.

The pPT0187 plasmid used in the experiments presented herein is the same coding sequence as the pPT0183, with the pPT0187 having 4 repeating units, i.e. monomer unit (ₙ), instead of the 6 repeating units of the pPT0183 plasmid. The size of the pPT0187 plasmid is approximately 6 kbp, which comprises about 4 kbp for the backbone and 2 kbp for the insert. Although the pPT0187 is used for the experiments presented herein, it should be apparent to a person of skill in the art that use of other plasmid DNA with silk gene protein are contemplated by the methods of the present invention. For example, any plasmid DNA with similar coding sequence as the pPT0183 is expected to produce similar results as those presented herein.

Experimental Results:

The plasmid DNA pPT0187 was modified as described herein to facilitate tethering of the silk protein to the surface of cancer cells once produced. The theory is that if the silkworm protein contained several Arg-Gly-Asp (RGD) attachment sites, it would constitute a mechanism for cell adhesion.

The modifications comprises solubilizing Lyophilized plasmid DNA pPT0187 in 10 mM Tris-EDTA then it was used to transform competent cells of E. coli DH5α and selected for growth on LB media containing Kanamycin (50 μg/mL).

Plasmid DNA from individual colonies was analyzed for inserts containing the SE coding gene by digestion with a combination of restriction endonucleases (REN): EcoRV-EcoR1, EcoRV-BamH1 and EcoRV-Xcm1 and electrophoresis on agarose gel.

Plasmid DNA from pPT0187 was digested with EcoRV and Bam HI RENs to remove the SE, the ends were filled with DNA polymerase, then the plasmid was re-ligated.

The product of the ligation was used to transform E. coli t DH5α.

Then the plasmid DNA was purified from colonies obtained and verified that the SE portion of the plasmid was removed. One of the plasmid obtained was labeled pPT0187e, which was used subsequently as the negative control. This pPT0187e plasmid lacked the silkworm gene, i.e. empty vector.

A technology routinely used in the study of cancer cells is the enforced expression of a gene contained in a bacterial expression vector (plasmid DNA). The process is termed transfection.

Plasmid pPT0187 and pPT0187e were transfected into human triple negative breast cancer cells, MDA231-LM2-4175 (LM2), utilizing the Amaxa nucleofector 2b system. Unless otherwise indicated, the cells were transfected at a ratio of 1 μg of DNA/1×10⁶ LM2 cells.

Two sets of experiments were conducted in vitro over separate periods. The experiments comprised transfecting plasmid pPT0187 in human triple negative breast cancer cells to determine if their growth (replication in culture) would be affected. As indicated in the tables below, the results provide proof that cancer cell growth is impaired when enforced to harbor plasmid pPT0187.

TABLE 1
First Set of Experiments.

| Experiment | Days after | | | | % Growth |
|---|---|---|---|---|---|
| No. | transfection | Sham | pPT0187e | pPT0187 | inhibition |
| 1 | 3 | (1) 1.6e6 | (1) 2.0e6 | (1) 1.05e6 | 49.5 |
| | | (2) 1.4e6 | (2) 1.96e6 | (2) 1.05e6 | |
| | | (3) 1.5e6 | (3) 1.97e6 | (3) 8.9e5 | |
| | | Av 1.5e6 | Av 1.97e6 | Av 996,666 | |
| 2 | 3 | Av 3.1e6 | Av 3.0e6 | Av 1.8e6 | 40 |
| 3 | 3 | (1) 3.5e5 | (1) 3.7e5 | (1) 2.0e5 | 46 |
| | | (2) 3.8e5 | (2) 3.8e5 | (2) 1.9e5 | |
| | | (3) 3.2e5 | (3) 3.6e5 | (3) 2.3e5 | |
| | | Av 320,000 | Av 360,000 | Av 230.000 | |
| 3-1 | 11 | Av 2.74e5 | Av 2.12e5 | Av 7.9e4 | 62.7 |

TABLE 2
Second Set of Experiments.

| Experiment | Days after | Dose of | % Growth |
|---|---|---|---|
| No. | transfection | plasmid | inhibition |
| 1 | 4 | 1 μg | 38 |
| 2 | 3 | 1 μg | 44 |
| 3 | 4 | 1 μg | 67 |
| 4(a) | 3 | 1 μg | 40 |
| 4(b) | 3 | 5 μg | 72 |

Each experiment consisted in three groups: cells transfected with the pPT0187 plasmid; cells transfected with the plasmid lacking the silkworm gene (empty vector), pPT0187e; and cells sham transfected. Each group consisted in three replicate cultures run simultaneously. The percentage growth inhibition is calculated by the formula: [(average number of cells treated with the empty vector−average number of cells treated with the pPT0187 plasmid)/average number of cells treated with the empty vector]×100.

These experimental results show that transfection with plasmid pPT0187 impairs the growth of human triple negative breast cancer cells in a reproducible and consistent way relative to controls.

Whereas all experiments are short-term experiments (the doubling time of the cancer cells used is 36-48 hours), they all show that impairment of cancer cell growth builds over the cell doubling time and is not due to non-specific toxic effects that would cause rapid death of the cells.

The effect on cancer cells is dose dependent, i.e., more pPT0187 plasmid leads to a greater effect, as illustrated in Table 2. For example, the experiment 4 results show that when 5 μg of pPT0187 (i.e. 4(b)) is transfected as compared to 1 μg (i.e. 4(a)), the percentage reduction increases drastically from about 40% to about 72%.

In Experiment Nos. 4(a) and 4(b) of the second set of experiments, a flow cytometric technique was used to determine the percentage of cells treated with the pPT0187 plasmid that actually show sign of cell death. The results show that approximately 20% of treated cells have also lost viability, i.e. are either dead or on their way to dying. This is consistent with the idea that plasmid pPT0187 once shuttled inside cancer cells causes their progressive decay, characterized by a mixture of inability to replicate and incipient cell death.

The methods described herein comprises direct injection into cancerous tumors. A further modification of this approach would be for targeting a cancerous tissue. For example, one approach would be to conditionally link the expression of the gene coding for the silk protein to the presence of the telomerase enzyme in the cancer cells. Since telomerase hallmarks cancer cells, linking the expression of the silk gene to the presence of telomerase adds specificity to the treatment to avoid off target effects. Thus, cancer cells could be specifically targeted without affecting normal cells.

The methods of the present invention provide a rational and effective approach to treat cancer by causing its demise by effectively reducing or completely eliminating nutrient supply vehicle through blood vessels and lymphatics. In addition, this approach also depletes nutrients present in the extracellular space, such as minerals, like iron, which promotes cell growth.

The methods of this invention is based on a testable, effective method to deliver the silk gene to cancer cells. It has already been demonstrated that mammalian cells can be transfected with the gene coding for spider silk fibers and produce silk fibers through a process which mimics the synthetic machinery present in the ampullate gland of orb-weaver spinning spiders, or in silk worms, as originally demonstrated by Lazaris et al (Science, 2002).

Specifically, Lazaris, et al., demonstrated that recombinant silk protein could be successfully produced in mammalian cells using a silk gene not greater than 60 kilodaltons. One or more embodiments of the present invention are based on transfecting cancer cells with a silk gene of predefined size but not bigger than 60 kilodaltons.

While the invention herein disclosed has been described by means of specific embodiments and applications thereof, numerous modifications and variations could be made thereto by those skilled in the art without departing from the scope of the invention set forth in the claims.

What is claimed is:

1. A method to treat solid cancer tissue such as breast, prostate, liver, renal, lung, pancreatic, ovarian, cervical, thyroid, melanoma, and sarcoma using a plasmid comprising:
selecting a patient with breast, prostate, liver, renal, lung, pancreatic, ovarian, cervical, thyroid, melanoma, or sarcoma cancerous tissue; and
administering a therapeutic dose of a plasmid in the form of an expression vector to said cancerous tissue, wherein the plasmid comprises a nucleic acid encoding the silk protein gene.

2. The method of claim 1, wherein the silk coding gene is physically linked with a transmembrane domain sequence.

3. The method of claim 1, wherein the silk coding gene is physically linked with one or multiple repeats of the RGD motif

4. The method of claim 1, wherein the silk coding gene is physically linked with a transmembrane domain sequence and the nucleic acid coding for one or multiple repeats of the RGD motif.

5. The method of claim 1, wherein said administering the plasmid to said cancer tissue comprises direct intra tumor injection of the expression vector.

6. The method of claim 1, wherein said administering the plasmid to said cancer tissue comprises intravenous injection of the expression vector.

7. The method of claim 1, wherein said administering the plasmid to said cancer tissue comprises application of the expression vector using dermal patch.

8. The method of claim 1, wherein the plasmid vector is in the form of a liposome, exosome, or any type of chemically synthesized or biologically derived small vesicles.

9. The method of claim 1, wherein the plasmid comprises the same coding sequence as the pPT0183.

10. The method of claim 1, wherein the plasmid comprises the same coding sequence as the pPT0183 with different number of repeats of the monomer unit.

11. A method to treat solid cancer cells such as breast, prostate, liver, renal, lung, pancreatic, ovarian, cervical, thyroid, melanoma, and sarcoma using a plasmid comprising:
selecting a patient with breast, prostate, liver, renal, lung, pancreatic, ovarian, cervical, thyroid, melanoma, or sarcoma cancer cells; and
administering a therapeutic dose of a plasmid in the form of an expression vector to said patient, wherein the plasmid comprises a nucleic acid encoding the silk protein gene.

12. The method of claim 11, wherein the silk coding gene is equal or less than 60 kilodaltons.

13. The method of claim 11, wherein the silk coding gene is physically linked with a transmembrane domain sequence.

14. The method of claim 11, wherein the silk coding gene is physically linked with one or multiple repeats of the RGD motif

15. The method of claim 11, wherein the silk coding gene is physically linked with a transmembrane domain sequence and the nucleic acid coding for one or multiple repeats of the RGD motif.

16. The method of claim 11, wherein said administering the plasmid to said cancer tissue comprises direct intra tumor injection of the expression vector.

17. The method of claim 11, wherein said administering the plasmid to said cancer tissue comprises intravenous injection of the expression vector.

18. The method of claim 11, wherein the plasmid vector is in the form of a liposome, exosome, or any type of chemically synthesized or biologically derived small vesicles.

19. The method of claim 11, wherein the plasmid comprises the same coding sequence as the pPT0183.

20. The method of claim 11, wherein the plasmid comprises the same coding sequence as the pPT0183 with different number of repeats of the monomer unit.
